(19) United States
(12) Patent Application Publication
(10) Pub. No.: US 2007/0010721 A1
(43) Pub. Date: Jan. 11, 2007

(54) Apparatus and system of Internet-enabled wireless medical sensor scale

(76) Inventors: Thomas C. H. Chen, Houston, TX (US); Connie J. Chen, Houston, TX (US)

Correspondence Address: Thomas C. H. Chen, 5468 Cedar Creek Dr., Houston, TX, 77056, US

(21) Appl. No.: 11/474,667

(22) Filed: Jun. 26, 2006

Related U.S. Application Data
(60) Provisional application No. 60/694,790, filed on Jun. 28, 2005.

(51) Int. Cl. A61B 5/00; G06Q 10/00

(52) U.S. Cl. 600/300; 128/920; 128/903; 705/2

(57) ABSTRACT

An Internet-Enabled Wireless Medical Sensor Scale System comprising of a) a plurality of Wireless Medical Sensor Scale Apparatus 1000 that can be placed any location where a wireless communication network is reachable and consists of a system processing unit 100 including microcontroller 110, system memory 120, flush memory 130 and LCD display 140 for processing vital sign related data, a multi-modal active sensor unit 300 for sensing, measuring and processing vital sign related data using process-based, bio-based and radio frequency identification (RFID) integrated sensor circuitry, a two-way wireless communication unit 200 using wireless communication strip antennas for transmitting vital sign related data to a remote server, and receiving commands from the said server for further processing instructions, a plurality of active foot sensing pad for sensing human body vital sign, a plurality of active health sensing substrate for sensing substance from human body, a configuration button 500, a operation button 600, a power source unit 900, b) a Web-based Personal Health Monitoring System, c) a Web-based Medical Care Monitoring System, and d) a system software 800 residing in microcontroller 110 for communicating with personal health and medical care monitoring systems through wireless communication network and Internet connection.

### BACKGROUND

1. Field of Invention

This invention relates in general to medical sensors, and in particular to an Internet-enabled Wireless Medical Sensor Scale apparatus and system that can be used for sensing, measuring and processing of personal medical, health and fitness related data, and transmitting these data automatically to a remote server, through wireless communication network and Internet connection, for data storage, monitoring and alerting purposes.

2. Description of Prior Art

Medical sensor is well understood in the art. It can be used to measure a person's relevant vital parameters, such as blood pressure, glucose concentration, heart rate and body temperature. Patents related to medical sensors can be classified into two major areas: 1) application specific medical sensors, and 2) general purpose medical sensors.

In application specific medical sensor area, different vital sign measurement, data collection and reporting methods are disclosed in the prior art. For example, in U.S. Pat. No. 5,419,321, Peter D. Evans describes an apparatus for non-invasive quantitative measurement of a substance in living tissue. In another example, U.S. Pat. No. 4,248,239 to Robert H. Ricciardelli reveals a polarized sensor assembly for a polarographic oxygen sensor. Another relevant example is U.S. Pat. Nos. 5,413,099 and 5,413,102, both to Schmidt et al., explain a medical sensor for monitoring vital signs in particular oxygen saturation. A further example is U.S. Pat. No. 5,261,892 to Bertaud et al. explains a device for storing and delivering a sensor through a catheter. Some patents of the prior art are related to implantable medical sensors. For example, in U.S. Pat. No. 6,201,980, Christopher B. Darrow, et al. describes an implantable chemical sensor system for medical application, which permits selective recognition of an analyte using an expandable biocompatible sensor. In other example, U.S. Pat. No. 6 6,106,475 to Low et al. discusses a device for use in placing a non-sterile sensor probe, such as an ultrasound scanning transducer, in a desired position within a patient's body. Some medical sensor patents are related to wearable medical sensors. For example, U.S. Pat. No. 6,755,795 to George Marmaropoulos reveals a wearable garment including medical sensor devices of well-know design that are selectively pressed against the skin of the wearer when it is desired to obtain medical reading. In another example, U.S. Pat. No. 6,579,231, Eric T. Phipps discloses a personal medical monitoring unit and system that includes a portable unit worn by a subject.

In general purpose medical sensor area, some patents in the prior art discuss how sensor devices are used to measure, monitor medical data, and managing these data through communication networks. For example, U.S. Pat. No. 6,847,294 to Wei-Kang Lin et al. discloses a radio medical monitoring system and method to use this system. Another example, U.S. Pat. No. 6,607,480, Ralf Bousseljot et al. reveals diagnostic information from the signals and data of medical measurement system without first reducing the measurement data to individual characteristics and then associating these using decision trees in order to form a diagnostic conclusion. Similarly, U.S. Pat. Nos. 5,645,059, 5,779,630 and 6,044,283, all to Michael E. Fein et al., disclose an encoding mechanism for a medical sensor which uses a modulated signal to provide the coded data to a remote analyzer. In another example, U.S. Pat. No. 6,875,174 to Jeffrey C. Braun, et, al. discloses a general-purpose, low-cost system provides comprehensive physiological data collection, with extensive data object oriented programmability and configurability for a variety of medical as well as other analog data collection applications. Other examples of related to general purpose medical sensors in the prior art include U.S. Pat. No. 6,074,345 to Johannes H. van Oostrom et al. disclosing a method and apparatus for connecting to and coordinating data communication of various medical devices having different communication protocols, U.S. Pat. No. 5,560,355 to Adman I. Merchant et al. revealing a medical sensors for detecting a blood characteristics, and U.S. Pat. No. 7,048,687 to James L. Reuss et al. discussing a limited use medical probe.

In reviewing above patents in the prior art, no disclosures directly related to the apparatus and system of Internet-Enabled Wireless Medical Sensor Scale of this invention are found.

### OBJECTS OF THE INVENTION

It is therefore an object of this invention to provide a medical sensor scale, different from traditional weight scale, that has a system processing unit, which includes microcontroller, system memory, flush memory and LCD display for processing and transmitting vital sign related data of human body.

It is also an object of this invention to provide a medical sensor scale that has a multi-modal active sensor unit for sensing, measuring and processing vital sign related data of human body. Multi-modal active sensor unit will include process-based sensor circuitry, bio-based sensor circuitry, and radio Frequency Identification (RFID) integrated sensor circuitry for sensing and analyzing vital sign of human body using signal reference.

It is another object of this invention to provide a medical sensor scale that has a two-way wireless communication unit for transmitting vital sign related data to a remote server, and receiving commands from the server further processing instructions. Two-way wireless communication unit will include Wi-Fi wireless communication strip antenna that can be used to communicate with LAN (Local Area Network) Access Point (A) of a near by Wi-Fi network system. two-way wireless communication unit will also include Bluetooth wireless communication strip antenna that can be used to communicate with the Piconet of a near by Bluetooth network system.

Another object of this invention is to provide a medical sensor scale with active foot sensing pad on the top of the medical sensor scale for sensing vital sign of human body to be used by above-mentioned process-based sensor circuitry.

Another object of this invention is to provide a medical sensor scale with active health sensing substrate on the top of medical sensor scale for sensing vital sign of human body to be used by above-mentioned bio-based sensor circuitry.

It is another object of this invention to provide a system that can transmit personal medical, health and fitness related data, automatically to a remote server, through wireless communication network and Internet connection, for personal health and medical care monitoring purposes.

Further objects and advantages of this invention will become apparent from a consideration of the ensuing descriptions and accompanying drawings.

### BRIEF DESCRIPTION OF THE DRAWINGS

For a further understanding of the objects and advantages of the present invention, references should be made to the following drawings in conjunction with the accompanying descriptions, wherein:

FIG. 1 is an illustrative block diagram of the components of Wireless Medical Sensor Scale Apparatus of the Internet-Enabled Wireless Medical Sensor Scale System of this invention.

FIG. 2 is an illustrative block diagram of the process-based sensor circuitry of Wireless Medical Sensor Scale Apparatus of this invention.

FIG. 3 is an illustrative block diagram of the bio-based sensor circuitry of Wireless Medical Sensor Scale Apparatus of this invention.

FIG. 4 is an illustrative block diagram of the top view of Wireless Medical Sensor Scale Apparatus with system and sensor display panel, display panel configuration button, active food sensing pad, active health sensing substrate, and scale operation control button of this invention.

FIG. 5 is an illustrative block diagram on the back of the side view of Wireless Medical Sensor Scale Apparatus with wireless communication strip antenna of this invention.

FIG. 6 is an illustrative block diagram of the back view of Wireless Medical Sensor Scale Apparatus with power source unit of this invention.

FIG. 7 is an illustrative diagram of the detail information on System and Sensor Display Panel of Wireless Medical Sensor Scale Apparatus of this invention.

FIG. 8 is an illustrative diagram of the communication interface between multiple Internet-Enabled Wireless Medical Sensor Scale Apparatus, Web-based Personal Health Monitoring System and Web-based Medical Care Monitoring System, through wireless communication and Internet connection, of this invention.

FIG. 9 is an illustrative diagram of the Application Tasks of System Software of Wireless Medical Sensor Scale Apparatus of this invention.

FIG. 10 is system diagram that illustrates the system flow of Web-based Personal Health Monitoring System of this invention.

FIG. 11 is a system diagram that illustrates the system flow of Web-based Medical Care Monitoring System of this invention.

FIG. 12 is a flow chart illustrating the administration and management process flow of Intelligent Controller of this invention.

FIG. 13 is a flow chart illustrating the process flow of Active Sensor Data Acquisition Task of this invention.

FIG. 14 is a flow chart illustrating the process flow of HTTP Request Processing Task of this invention.

FIG. 15 is a flow chart illustrating the process flow of Two-Way Wireless Communication Task of this invention.

FIG. 16 is a flow chart illustrating the process flow of Configuration Button Task of this invention.

FIG. 17 is a flow chart illustrating the process flow of Operation Button Task of this invention.

FIG. 18 is a flow chart illustrating the process flow of LCD Display Task of this invention.

### REFERENCE NUMERALS

100 System Processing Unit
110 Microcontroller
120 System Memory
130 Flush Memory
140 LCD Display
200 Two-way Wireless Communicaiton Unit
250 Wireless Communication Strip Antenna
251 Wi-Fi Wireless Communication Strip Antenna
252 Bluetooth Wireless Communication Strip Antenna
300 Multi-Modal Acive Sensor Unit
301 Active Sensor
302 Transducer
311 Process Sensor
312 Transducer
313 Signal Filter
314 Signal Multiplexer
315 Process Signal Reference
316 Amplifier
317 Analog-to-Digital Converter
321 Biocatalyst
322 Transducer
323 Signal Filter
324 Signal Multiplexer
325 Biocatalyst Signal Reference
326 Amplifier
327 Analot-to-Digital Converter
400 Power Source Unit
400 Power Source Light
500 Configuration Button
600 Operation Button
710 System and Sensor Display Panel
711 System Configuration Function Panel
712 Sensor Configuration Functional Panel
713 Sensor Measuring Data Display Panel
714 User Defined Data Display Panel
715 User Identification Scale Location, Time and Date Display Panel
720 Display Panel Configuration Button
730 Active Foot Sensing Pad Time and Date Display Panel
740 Active Health Sensing Substrate
750 Scale Operation Control Button
800 System Software
810 Intelligent Controller
811 Task Control Administrator
820 Active Sensor Data Acquisition Task
821 HTTP Request Processing Task
822 Two-Way Wireless Communication Task
823 Configuration Button Task
824 Operation Button Task
825 LCD Display Task
830 Real-Time Operating System (RTOS)
900 Power Source Unit
1000 Wireless Medical Sensor Scale
2000 Web-based Personal Health Monitoring System
3000 Web-based Medical Care Monitoring System

### PREFERRED EMBODIMENT—APPARATUS

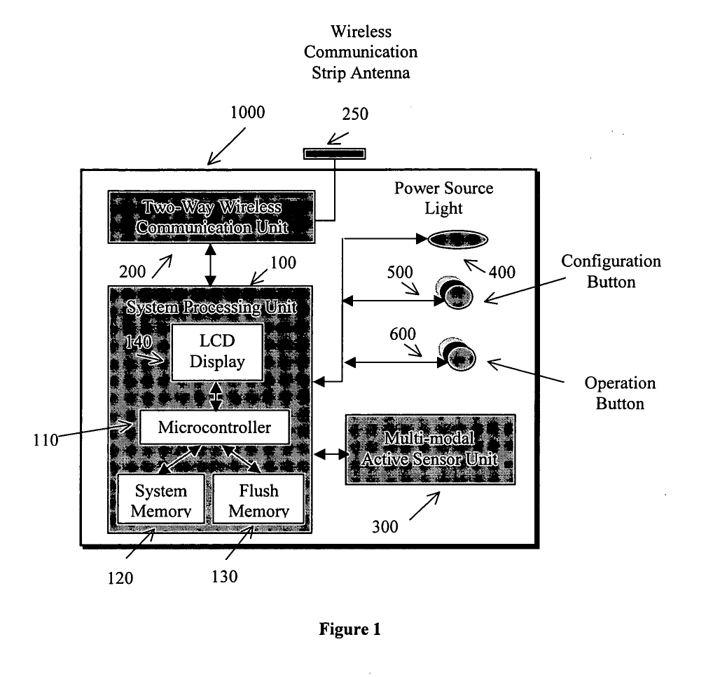

The key component of the Internet-Enabled Wireless Medical Sensor Scale System is Wireless Medical Sensor Scale Apparatus 1000 described in this patent, as showing in FIG. 1. The major component of Wireless Medical Sensor Scale Apparatus 1000 is System Processing Unit 100 that includes Microcontroller 110, System Memory 120, Flush Memory 130, and LCD Display 140. System Processing Unit 100 is also connected to Two-Way Wireless Communication Unit 200 and Multi-modal Active Sensor Unit 300, Operation Button 600, Configuration Button 500, and Power Source Light 400. System Processing Unit 100 includes a System Software 800 that contains a Web server and an embedded intelligent agent server for ubiquitous recording, monitoring and alerting on the dynamics of parameter measurements.

Two-Way Wireless Communication Unit 200 is connected to Wireless Communication Strip Antenna 250 for two-ways wireless communications. Different communication strip antennas, Wi-Fi wireless communication strip antenna and Bluetooth wireless communication strip antenna, are used in this patent.

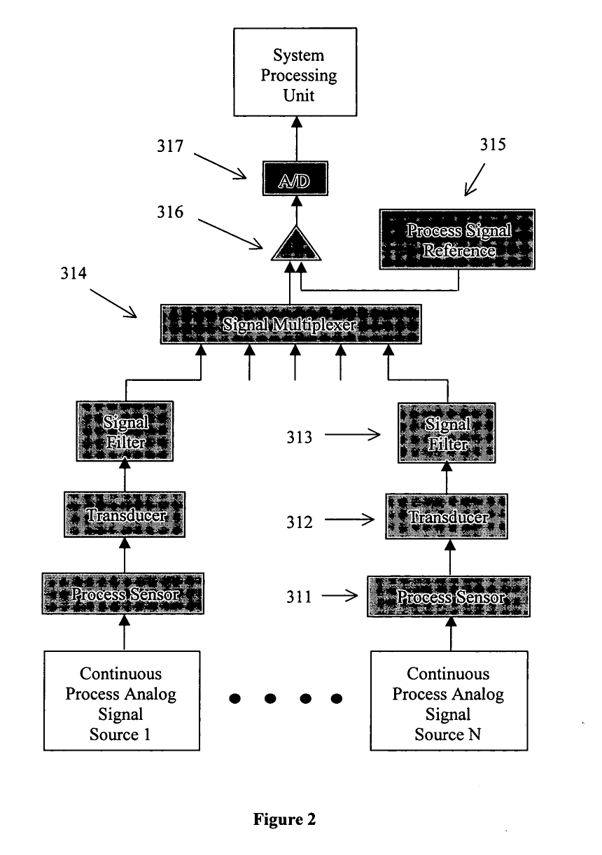

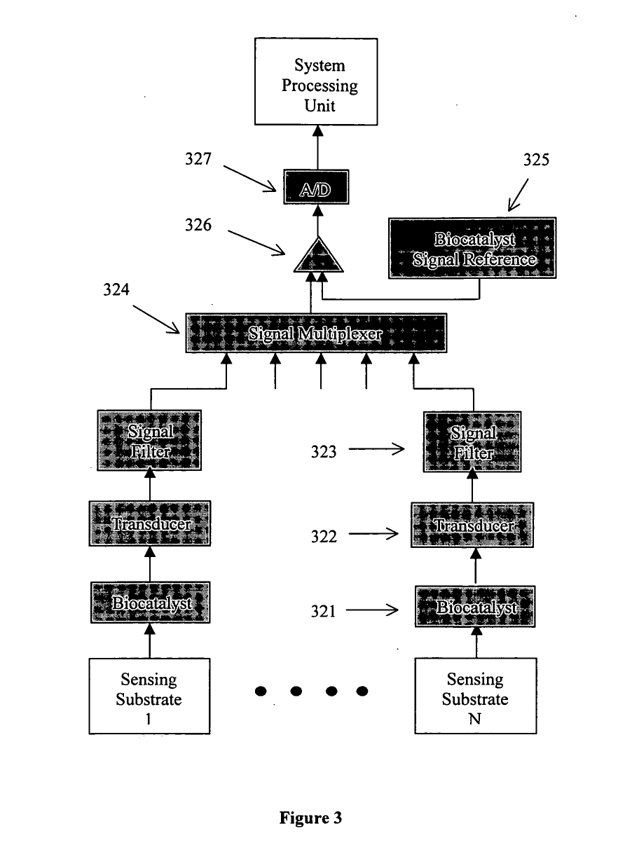

Multi-modal Active Sensor Unit 300 includes process-based sensor circuitry, bio-based sensor circuitry, and radio Frequency Identification (RFID) integrated sensor circuitry. Each circuitry specializes in detecting a specific medical, health and fitness related parameters, such as body temperature, body weight, body mass index, and body fat. FIG. 2 shows a process-based sensor circuitry, which includes Process Senor 311, Transducer 312, Signal Filter 313, Signal Multiplexer 314, Process Signal Reference 315, Signal Amplifier 316, and A/D Converter 317. FIG. 3 shows a bio-based sensor circuitry, which includes Biocatalyst 321, Transducer 322, Signal Filter 323, Signal Multiplexer 324, Biocatalyst Signal Reference 325, Signal Amplifier 326, and A/D Converter 327.

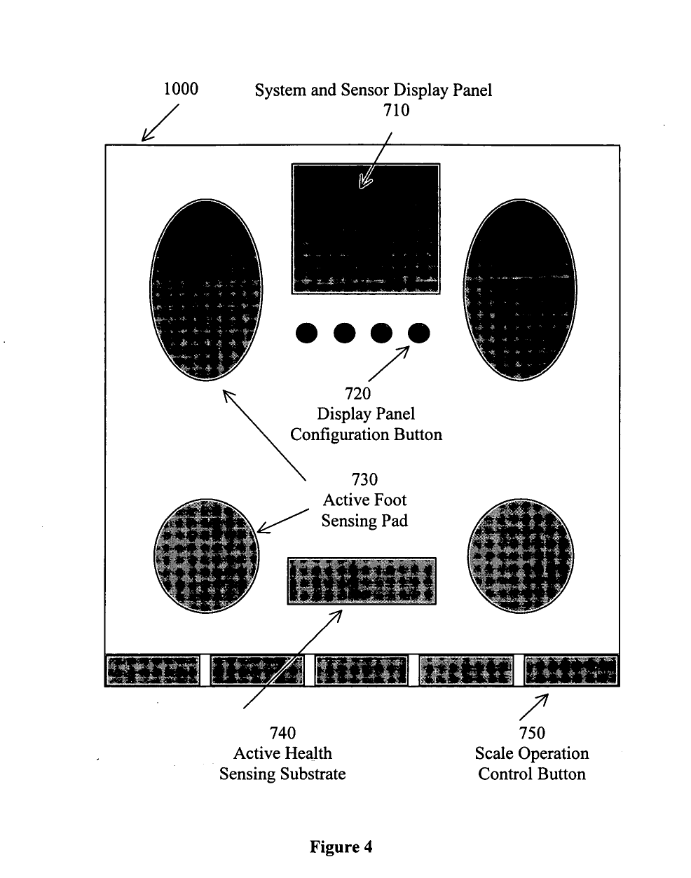

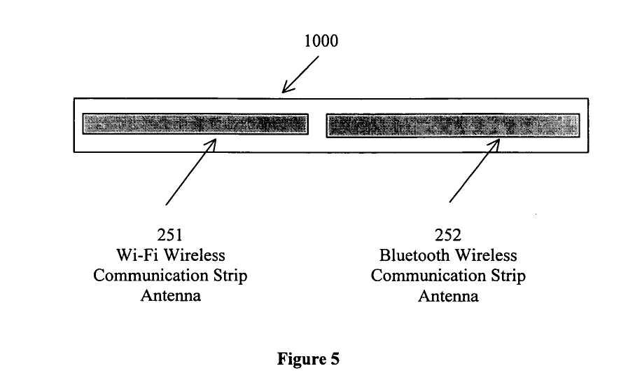

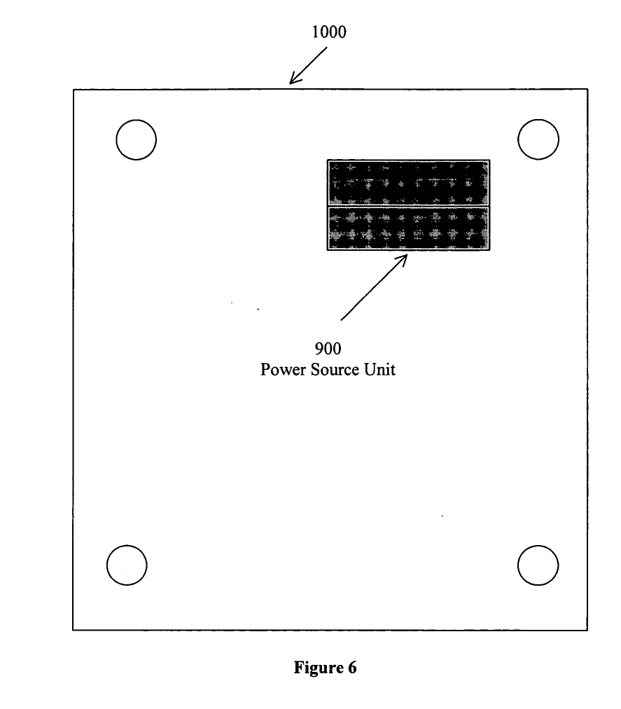

FIG. 4 shows the top view of Wireless Medical Sensor Scale Apparatus 1000 of this invention, which includes System and Sensor Display Panel 710, Display Panel Configuration Button 720, Active Foot Sensing Pad 730, Active Health Sensing Substrate 740, and Scale Operation Control Button 750. FIG. 5 shows on the back of the side view of Wireless Medical Sensor Scale Apparatus 1000 of this invention, which includes a Wi-Fi Wireless Communication Strip Antenna 251 and a Bluetooth Wireless Communication Strip Antenna 252. FIG. 6 shows the back view of Wireless Medical Sensor Scale Apparatus 1000 of this invention, which includes a housing for Power Source Unit 900.

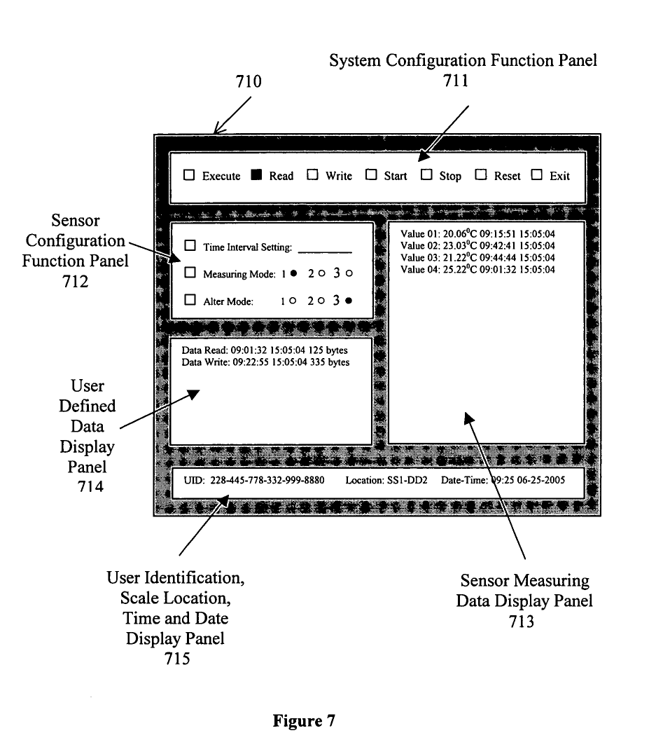

FIG. 7 shows the detail information on System and Sensor Display Panel 710 of this invention, which includes System Configuration Function Panel 711, Sensor Configuration Function Panel 712, Sensor Measuring Data Display Panel 713, User Defined Data Display Panel 714, and User Identification, Scale Location, Time and Date Display Panel 715.

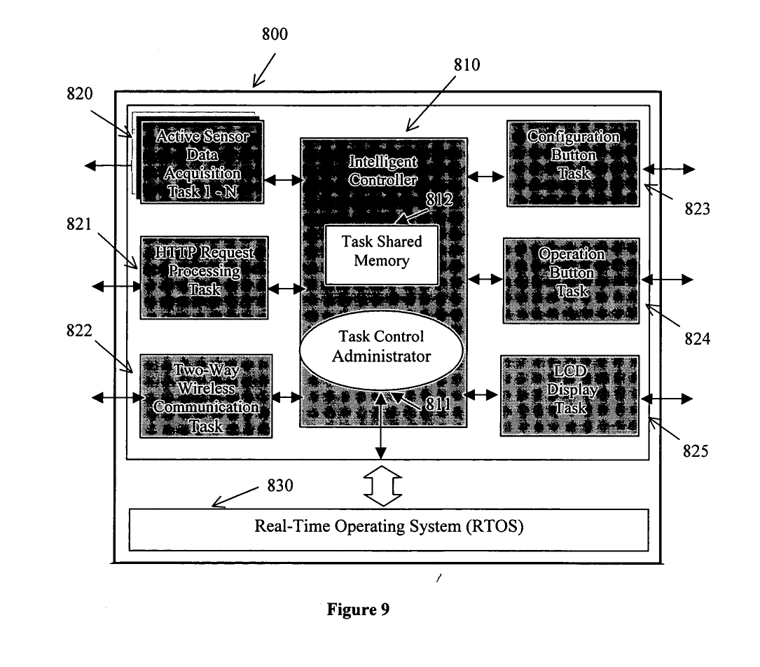

System Processing Units 100 contains System Software 800 which uses an active, real-time monitoring method to sense, measure and process of user's medical, health and fitness related parameters, and automatically transmitting these data to a remote server for data storage and monitoring purposes. FIG. 9 shows the Application Tasks of System Software 800, which includes Intelligent Controller 810 that comprises Task Control Administrator 811, and a plurality of Task Shared Memory 812. Task Control Administrator 811 manages and controls the execution of application tasks that include a plurality of Active Sensor Data Acquisition Task 820, HTTP Request Processing Task 821, Two-Way Wireless Communication Task 822, Configuration Button Task 823, Operation Button Task 824, and LCD Display Task 825.

### PREFERRED EMBODIMENT—SYSTEM AND OPERATION

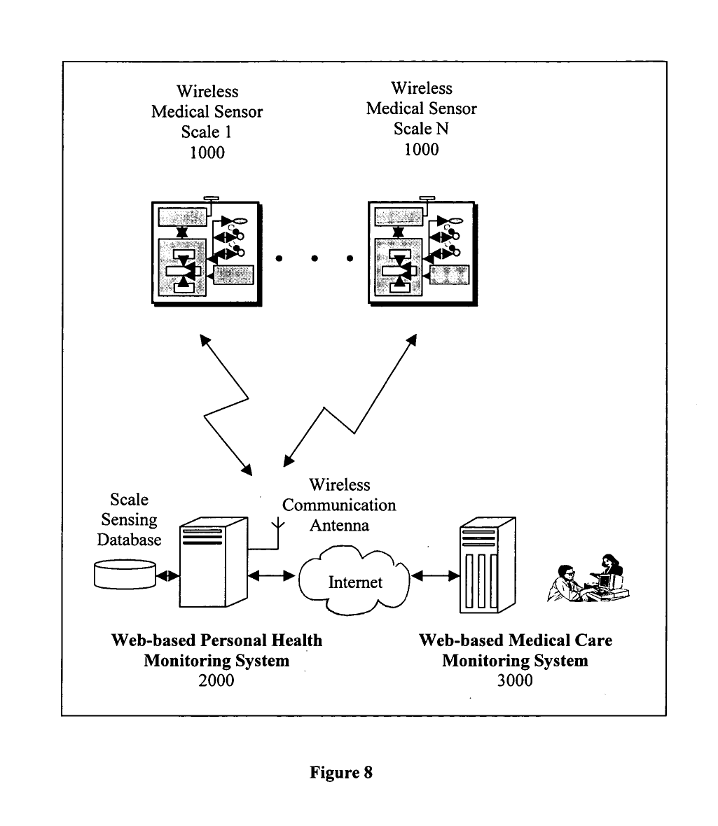

Internet-Enabled Wireless Medical Sensor Scale System of this invention consists of a plurality of Wireless Medical Sensor Scale Apparatus 1000, a Web-based Personal Health Monitoring System 2000, and a Web-based Medical Care Monitoring System 3000. Wireless Medical Sensor Scale Apparatus 1000 can be placed any location where a Wi-Fi or Bluetooth wireless communication network is reachable. FIG. 8 is an illustrative schematic diagram that shows the communication interface between multiple Wireless Medical Sensor Scale Apparatus 1000, Web-based Personal Health Monitoring System 2000 and Web-based Medical Care Monitoring System 3000, through wireless communication and Internet connection, of this invention.

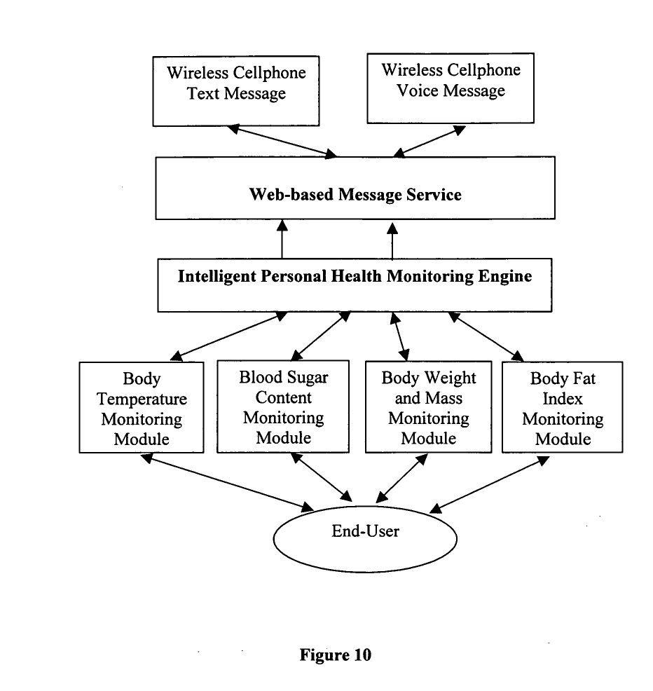

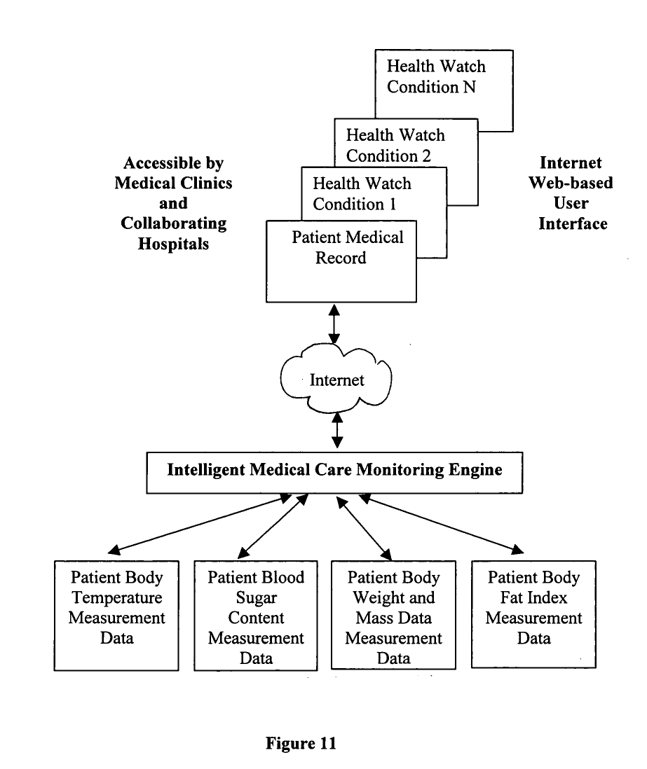

FIG. 10 is a system diagram that illustrates the system flow of Web-based Personal Health Monitoring System 2000. FIG. 11 is a system diagram that illustrates the system flow of Web-based Medical Care Monitoring System 3000.

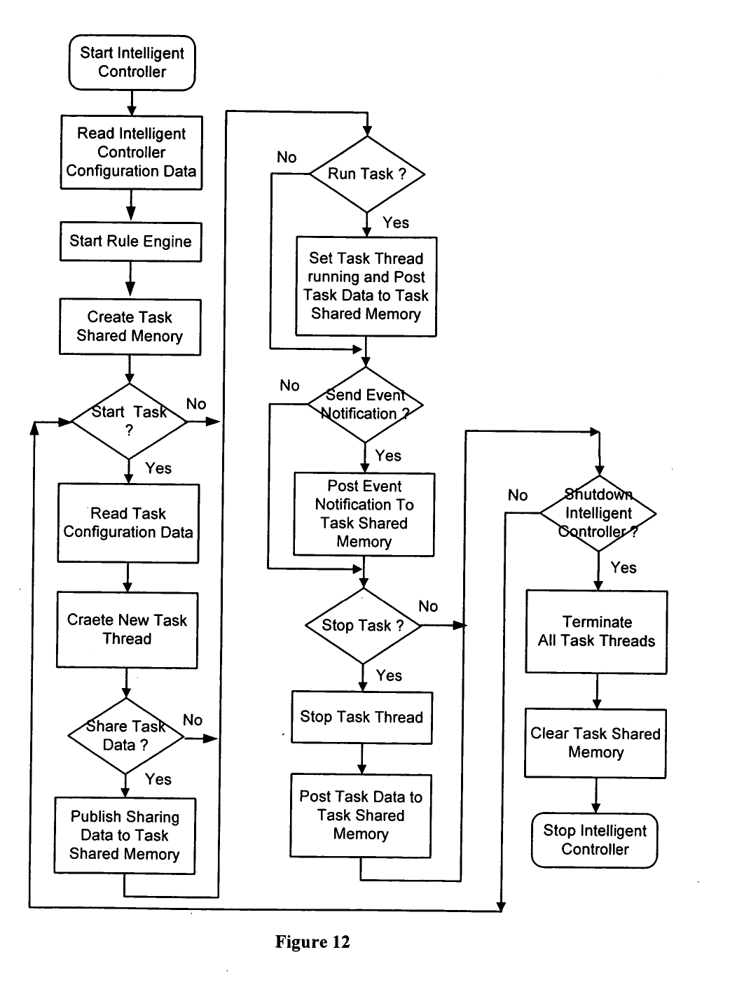

FIG. 12 is a flow chart illustrating the administration and management process flow of Intelligent Controller 810. At system startup, Real-Time Operating System (RTOS) 830 is first loaded into System Memory 120. Real-Time Operating System 830 then loads and starts Intelligent Controller 810. Intelligent Controller 810 first reads configuration data, which includes a set of control rules from Flush Memory 130 and writes the control rules to Task Shared Memory 812. Task Control Administrator 811 of Intelligent Controller 810 then starts the rule engine and uses the control rules to create a plurality of Task Shared Memory 812 to be used by all related tasks. Also using the control rules, Task Control Administrator 811 creates separate parallel execution thread for each Active Sensor Data Acquisition Task 820, and HTTP Request Processing Task 821, Two-Way Wireless Communication Task 822, Configuration Button Task 823, Operation Button Task 824, LCD Display Task 825. After the creation of execution threads, Task Control Administrator 811 starts and registers all running tasks at Task Shared Memory 812. Task Control Administrator 811 also publishes those tasks that can be used by other tasks at Task Shared Memory 812. During the task operation, Task Control Administrator 811 can stop or restart a thread for a particular task at the system's request or when triggered by different task conditions. At system shutdown, Task Control Administrator 811 stops all execution threads, and clears all entries at Task Shared Memory 812.

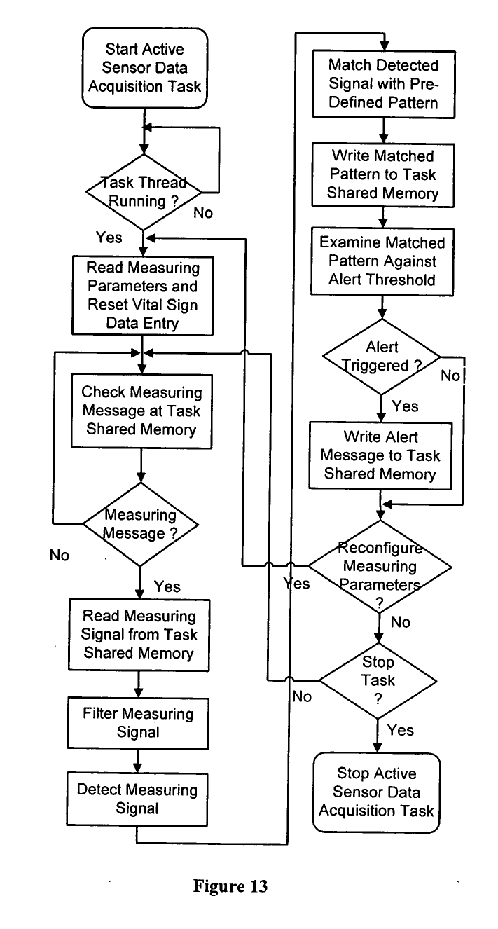

FIG. 13 is a flow chart illustrating the process flow of Active Sensor Data Acquisition Task 820. Active Sensor Data Acquisition Task 820 begins by reading the measurement parameters at Task Shared Memory 812 of System Memory 120 and re-setting all data entries. Active Sensor Data Acquisition Task 820 then checks for a measurement message at Task Shared Memory 812. If there is a measurement message waiting to be processed, Active Sensor Data Acquisition Task 820 first reads measurement signals at Task Shared Memory 812, and then filters the measurement signals to match the pre-defined pattern. The results of matched pattern are then written onto Task Shared Memory 812 for use by other related application tasks. Active Sensor Data Acquisition Task 820 continues to examine matched pattern against the alert threshold. If an alert is triggered, Active Sensor Data Acquisition Task 820 writes an alert message to Task Shared Memory 812. This alert message is later transmitted to a designated Website location through Two-Way Wireless Communication Task 822. The execution of Active Sensor Data Acquisition Task 820 continues until it has been terminated by Intelligent Controller 810.

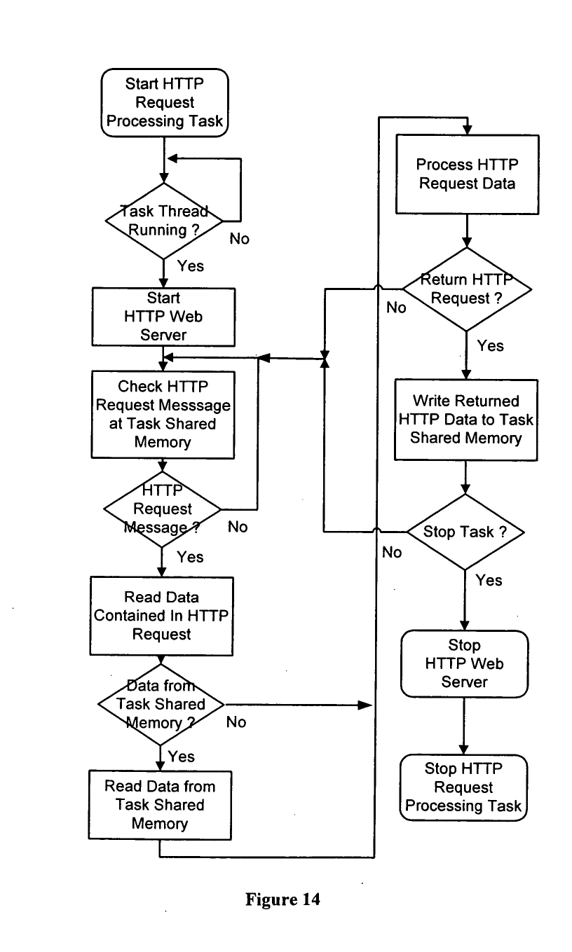

FIG. 14 is a flow chart illustrating the process flow of HTTP Request Processing Task 821. HTTP Request Processing Task 821 begins with the reading HTTP configuration data from Flush Memory 130. HTTP Request Processing Task 821 then starts the HTTP Web Server that contains port listeners and request handlers. Once HTTP Request Processing Task 821 is up and running, it checks the HTTP request message at Task Shared Memory 812. The HTTP request message is from Two-Way Wireless Communication Task 822. If there is a HTTP request at Task Shared Memory 812 waiting to be processed, HTTP Request Processing Task 821 reads the request, and then tries to validate the authorization and authentication of the request specified in the configuration data. After the request has been validated, HTTP Request Processing Task 821 reads data contained in the HTTP request, interrogates the data, and determines what actions must be taken. The action could be reading more data from Task Shared Memory 812 or posting data to Task Shared Memory 812. Permissions to take certain actions by HTTP Request Processing Task 821 are also specified in the HTTP configuration data. HTTP Request Processing Task 821 then writes a returned request message to Task Shared Memory 812. The returned request message is then transferred from Task Shared Memory 812 to Two-Way Wireless Communication Unit 200 by Two-Way Wireless Communication Task 822. The execution of HTTP Request Processing Task 821 continues until it has been terminated by Intelligent Controller 810.

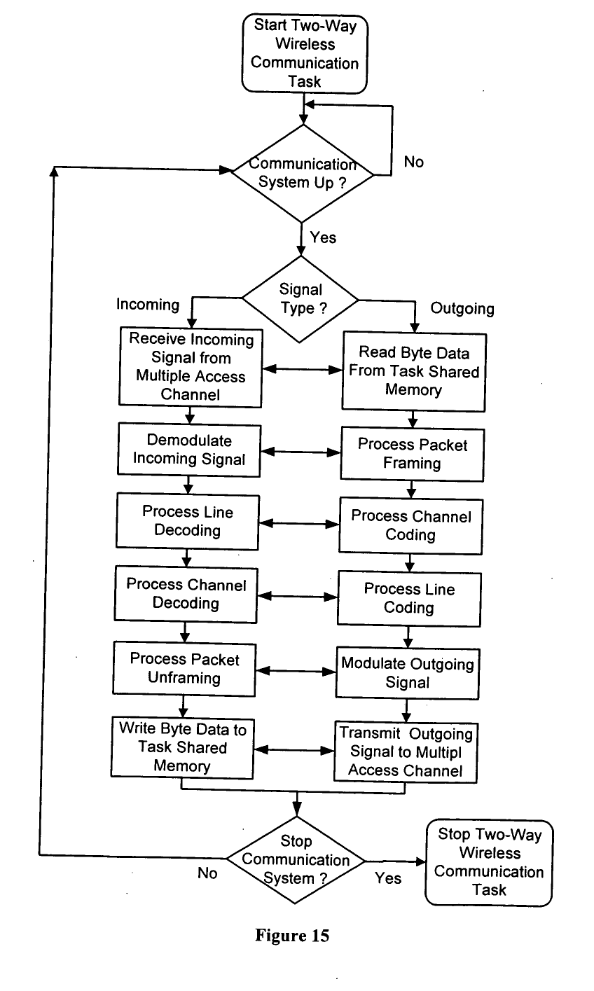

FIG. 15 is a flow chart illustrating the process flow of Two-Way Wireless Communication Task 822. Two-Way Wireless Communication Task 822 processes both incoming and outgoing signals from Two-Way Wireless Communication Unit 200 that is connected to Wireless Communication Strip Antenna 250. To process the incoming wireless communication signal, Two-Way Wireless Communication Task 822 first receives the incoming signal from the multiple access channel of Two-Way Wireless Communication Unit 200, and then writes it to Task Shared Memory 812. To process the outgoing wireless communication signal, Two-Way Wireless Communication Task 822 first reads byte data from Task Shared Memory 812, and then routes it to Two-Way Wireless Communication Unit 200 to be transmitted through the multiple access channel. The execution of Two-Way Wireless Communication Task 822 continues until it has been terminated by Intelligent Controller 810.

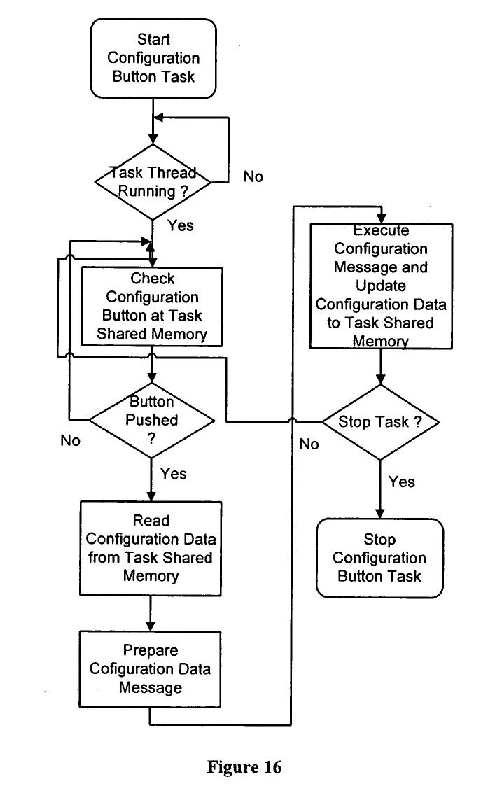

FIG. 16 is a flow chart illustrating the process flow of Configuration Button Task 823. Configuration Button Task 823 begins with the checking of configuration message at Task Shared Memory 812. When Configuration Button 500 is pressed, the system writes a configuration message onto Task Shared Memory 812. This message is read by Configuration Button Task 823 that then proceeds to read the scale location data and patient's medical, health, and fitness related data at Task Shared Memory 812 to prepare a configuration message. The configuration message is written onto Task Shared Memory 812 and updated by the system. The execution of Configuration Button Task 823 continues until it has been terminated by Intelligent Controller 810.

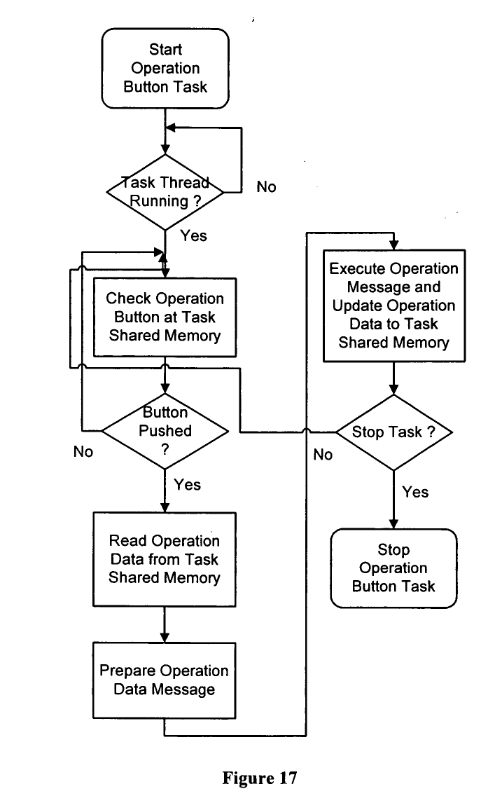

FIG. 17 is a flow chart illustrating the process flow of Operation Button Task 824. Operation Button Task 824 begins by checking the operation message at Processor Shared Memory 812. When Operation Button 824 is pressed, the system writes the operation message onto Task Shared Memory 812. The operation message is then performed by the system. The execution of Operation Button Task 824 continues until it has been terminated by Intelligent Controller 810.

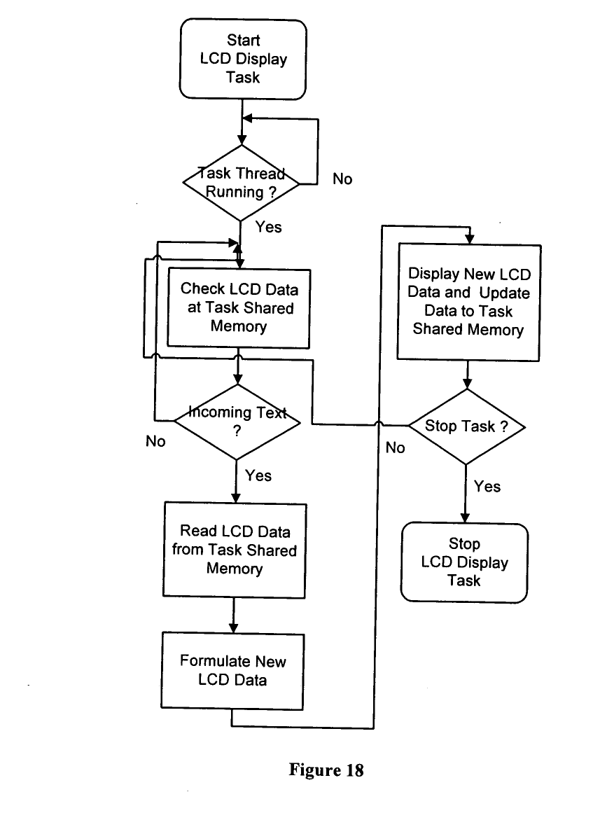

FIG. 18 is a flow chart illustrating the process flow of LCD Display Task 825. LCD Display Task 825 begins by checking the display message at Shared Memory 812. If there is a message at System Memory 810 waiting for display, the system will read the message and display it accordingly. The execution of LCD Display Task 825 continues until it has been terminated by Intelligent Controller 810.

What is claimed is:

1. An Internet-Enabled Wireless Medical Sensor Scale System comprising of a) a plurality of Wireless Medical Sensor Scale Apparatus that can be placed any location where a wireless communication network is reachable and said Wireless Medical Sensor Scale Apparatus consists of (i) a system processing unit including microcontroller, system memory, flush memory and LCD display for processing vital sign related data, (ii) a multi-modal active sensor unit for sensing, measuring and analyzing vital sign related data using process-based sensor circuitry, bio-based sensor circuitry and radio frequency identification (RFID) integrated sensor circuitry, (iii) a two-way wireless communication unit using wireless communication strip antennas for transmitting vital sign related data to a remote server, and receiving commands from the said server for further processing instructions, (iv) a plurality of active foot sensing pad for sensing human body vital sign to be used by said process-based sensor circuitry and said radio frequency identification (RFID) integrated sensor circuitry, (v) a plurality of active health sensing substrate for sensing substance from human body to be used by said bio-based sensor circuitry and said radio frequency identification (RFID) integrated sensor circuitry, (vi) a configuration button, (vii) a operation button, (viii) a power source unit, b) a Web-based Personal Health Monitoring System, c) a Web-based Medical Care Monitoring System, and d) a system software residing in said microcontroller which contains a plurality of task shared memory, and intelligent controller with a task control administrator to manage and control the execution of application tasks, and the said application tasks include active sensor data acquisition task, two-way wireless communication task, Hypertext Transmission Protocol (HTTP) request processing task, configuration button task, operation button task, and LCD display task, and the said intelligent controller uses an active, real-time monitoring method to measure and process vital signs for providing alert by transmitting a emergency request to remote monitoring station for immediate assistance, and the said system software includes a HTTP Web server that can respond to a remote request sent wirelessly from a remote monitoring station through a standard Internet browser, anywhere and anytime, and the said intelligent controller and all said application tasks of the said system software are running under separate system threads concurrently to fully utilize system processing power, and said system software can make two-way communications with said Web-based Personal Health Monitoring System and said Web-based Medical Care Monitoring System through wireless communication network and Internet connection.

2. The process-based sensor circuitry as recited in claim 1, comprising:
(a) a process sensor, and means for processing analog signal source data,
(b) a transducer, and means for converting physical measurement into alternative digital form,
(c) a signal bandpass filter, and means for band pass filtering signals to desired level,
(d) a signal multiplexer, and means for encodeing information from multiple signal sources into a single channel,
(e) a linear power amplifier, and means for amplifying signals to desired level,
(f) a analog-to-digital (A/D) converter, and means for converting analog data from said transducer to digital signal,
(g) a process signal reference, and means for converting analogy reading into digital data format based on process signal reference.

3. The bio-based sensor circuitry as recited in claim 1, comprising:
(a) a biocatalyst, and means for initiating a chemical reaction when exposed to substance from human body,
(b) a transducer, and means for converting physical measurement into alternative digital form,
(c) a signal bandpass filter, and means for band pass filtering signals to desired level,
(d) a signal multiplexer, and means for encodeing information from multiple signal sources into a single channle,
(e) a linear power amplifier, and means for amplifying signals to desired level,
(f) a analog-to-digital A/D) converter, and means for converting analog data from said transducer to digital signal,
(g) a biocatalyst signal reference, and means for converting chemical response reacted to substance from human body into digital data format based on biocatalyst signal reference.

4. The radio frequency identification (RFID) integrated sensor circuitry as recited in claim 1 is selected to use passive radio frequency identification (RFID) integrated sensor circuitry.

5. The radio frequency identification (RFID) integrated sensor circuitry as recited in claim 1 is selected to use active radio frequency identification (RFID) integrated sensor circuitry.

6. The wireless communication strip antenna as recited in claim 1 is selected to use Wi-Fi wireless communication strip antenna.

7. The wireless communication strip antenna as recited in claim 1 is selected to use Bluetooth wireless communication strip antenna.

8. The system software as recited in claim 1, comprising:
(a) a real-time operating system, and means for executing system start-up, memory configurations, input and output configurations, data file configurations, and system shutdown of said system processor,
(b) an intelligent controller, and means for administrating and managing said related application tasks of said system processor.

9. The intelligent controller as recited in (b) of claim 8, comprising:
(a) a task control administrator for administrating and managing said related application tasks,
(b) a plurality of task shared memory for storing and manipulating data entries of said related application tasks during system execution.

10. The task control administrator as recited in (a) of claim 9, comprising:
(a) a plurality of control rules, and means for describing control instructions of said related application tasks,
(b) a rule engine, and means for executing said control rules.

11. The intelligent controller as recited in (b) of claim 8 administrate and manage of said related application tasks of:
(a) an active sensor data acquisition task,
(b) a two-way wireless communication task,
(c) a hypertext transmission protocol (http) request processing task,
(d) a configuration button task,
(e) an operation button task,
(f) a LCD display task.

12. The intelligent controller as recited in (b) of claim 8, wherein said administrating and managing of said related application tasks, comprising the steps of:
(a) reading intelligent controller configuration and control rule data from said flush memory,
(b) starting rule engine,
(c) creating said task shared memory,
(d) checking running status of all tasks,
(e) reading task configuration data of a task if said task needs to be started,
(f) creating a task thread for said task,
(g) publishing data sharing option to said task shared memory of said task,
(h) setting said task running,
(i) updating data entry at said task shared memory of said task,
(j) writing event notification to said task shared memory of said task,
(k) repeating steps (d) through (j) while there are more tasks to be started,
(l) stopping said task at the request of said task control administrator,
(m) terminating all related application tasks, and clearing said task shared memory at the shutdown request of said task control administrator.

13. The active sensor data acquisition task as recited in (a) of claim 11, comprising the steps of:
(a) checking running status of said active sensor data acquisition task,
(b) reading measurement parameters,
(c) resetting vital sign signal data entry,
(d) checking measuring signal from said task shared memory,
(e) filtering said measurement signal,
(f) detecting said measurement signal,
(g) matching the detected signal with pre-defined reference pattern,
(h) writing matched pattern to said task shared memory,
(i) examining said matched pattern against an alert threshold,
(j) writing alert message to said task shared memory,
(k) re-reading said measurement parameters and re-setting said vital sign signal data entry if reconfiguration of said measurement parameters are needed,
(l) repeating steps (b) through (k) while there are said measuring message, and said active sensor data acquisition task is running,
(m) stopping said active sensor data acquisition task at request of said task control administrator.

14. The two-way wireless communication task as recited in (b) of claim 11, comprising the steps of:
(a) checking system status of said two-way wireless communication task,
(b) receiving incoming signal from multiple access channel,
(c) demodulating said income message using configurable demodulation routine,
(d) processing line decoding,
(e) processing channel decoding,
(f) processing packet un-framing,
(g) writing byte data to said task shared memory,
(h) repeating steps (b) through (g) while there is an incoming wireless signals,
(i) reading byte data from said task shared memory,
(j) processing packet framing of said byte data,
(k) processing channel coding,
(l) processing line coding,
(m) modulating outgoing signals using configurable modulation routine,
(n) transmitting said outgoing signals to said multiple access channel,
(o) repeating steps (i) through (n) while there is an outgoing wireless signals,
(p) stopping said two-way wireless communication task at the request of said intelligent controller.

15. The http request processing task as recited in (c) of claim 11, comprising the steps of:
(a) checking running status of said http request processing task,
(b) starting http web server,
(c) checking http request message at said task shared memory,
(d) reading data containing in http request from said task shared memory,
(e) reading data from said task shared memory if additional data is needed,
(f) processing said http request data,
(g) writing returned http request data to said task shared memory if returning http request is needed,
(h) repeating steps (c) through (g) while there are said http request messages at said task shared memory, and said http request task is running,
(i) stopping said http web Server and said http the request task at request of said task control administrator.

16. The configuration button task as recited in (d) of claim 11, comprising the steps of:
(a) checking running status of said configuration button task,
(b) checking on configuration button message at said task shared memory,
(c) reading configuration button status at said task shared memory,
(d) checking status value of said configuration button task,
(e) writing sleeping mode message to said task shared memory if status value is on,
(f) writing walking up message to said task shared memory if status value is off,
(g) repeating steps (b) through (f) while there are said configuration button messages at said task shared memory, and said configuration button task is running,
(h) stopping said configuration button task at the request of said task control administrator.

17. The operation button task as recited in (e) of claim 11, comprising the steps of:
(a) checking running status of said operation button task,
(b) checking on operation button message at said task shared memory,
(c) reading operation button status at said task shared memory,
(d) checking status value of said operation button task,
(e) writing sleeping mode message to said task shared memory if status value is on,
(f) writing walking up message to said task shared memory if status value is off,
(g) repeating steps (b) through (f) while there are said operation button messages at said task shared memory, and said operation button task is running,
(h) stopping said operation button task at the request of said task control administrator.

18. The LCD display button task as recited in (f) of claim 11, comprising the steps of:
(a) checking running status of said LCD display button task,
(b) checking on LCD display button message at said task shared memory,
(c) reading LCD display button status at said task shared memory,
(d) checking status value of said LCD display button task,
(e) writing sleeping mode message to said task shared memory if status value is on,
(f) writing walking up message to said task shared memory if status value is off,
(g) repeating steps (b) through (f) while there are said LCD display button messages at said task shared memory, and said LCD display button task is running,
(h) stopping said LCD display button task at the request of said task control administrator.
